(19) United States
(12) Patent Application Publication
(10) Pub. No.: US 2010/0130849 A1
(43) Pub. Date: May 27, 2010

(54) SYSTEM AND METHOD FOR PATIENT MOTION COMPENSATION DURING MAGNETIC RESONANCE SCANS

(75) Inventors: Xiaodong Tao, Niskayuna, NY (US); Thomas Kwok-Fah Foo, Clifton Park, NY (US); Sandeep Narendra Gupta, Clifton Park, NY (US)

Correspondence Address: GENERAL ELECTRIC COMPANY;GLOBAL RESEARCH, ONE RESEARCH CIRCLE, PATENT DOCKET RM. BLDG. K1-4A59, NISKAYUNA, NY, 12309, US

(73) Assignee: GE GLOBAL RESEARCH, SCHENECTADY, NY (US)

(21) Appl. No.: 12/624,504

(22) Filed: Nov. 24, 2009

Related U.S. Application Data
(60) Provisional application No. 61/118,114, filed on Nov. 26, 2008.

(51) Int. Cl. A61B 5/055

(52) U.S. Cl. 600/410

(57) ABSTRACT

A method for motion compensation includes acquiring an initial volumetric localizer to establish an initial object position and initial object orientation at an initial state, acquiring a fast localizer of the object at a present state, aligning the fast localizer to the initial volumetric localizers to determine object motion between the initial state and the present state, and modifying an imaging protocol using the object position and orientation at the present state.

### CROSS REFERENCE TO RELATED APPLICATIONS

This Non-provisional application hereby claims priority to U.S. Provisional Patent Application No. 61/118,114, entitled “SYSTEM AND METHOD FOR PATIENT MOTION COMPENSATION DURING MAGNETIC RESONANCE SCANS”, filed Nov. 26, 2008, which is herein incorporated by reference.

### BACKGROUND

Embodiments of the invention relate generally to imaging techniques and more particularly to a system and method for patient motion compensation during magnetic resonance scans.

In current Magnetic resonance imaging (MRI) acquisition processes, scout or localizer images of an object to be imaged are typically acquired before any diagnostic images are acquired. An operator reviews the localizer images and manually sets scanning parameters to acquire images of the object in a way that provides the most diagnostic or scientific values. Such MR imaging processes are very demanding on the operator requiring specific knowledge and skill. For example, it requires that the operator is able to recognize the patient orientation from the orthogonal views of the localizer images and determine the scan planes that are necessary to produce object images that conform to the standard views, or desired views. Furthermore, current MR imaging processes may also suffer from inconsistency between operators and between imaging sessions for the same operator.

Previous work in this area have addressed the problem of automatically computing and prescribing anatomically consistent scan planes using landmark based methods or statistical atlases. Although such approaches can be used to identify patient position and orientation and thereafter prescribe imaging planes automatically, the overall process including acquiring the volumetric data typically takes of the order of 40 seconds to more than a minute to perform. Accordingly, motion during an exam remains a major problem in most imaging modalities and particularly in MRI. Even if an automated scheme is used at some stage of an exam for automatic scan plane planning, if the subject subsequently moves from this calculated position, all the computed planes will be incorrect and will not yield the correct desired anatomy. Repeating the full automatic scan plan planning system many times during an exam to re-compute new scan planes, although possible, is often not feasible in practice due to the amount of time this would require.

What is needed is a fast method to quickly refine or update the scan planes previously computed in the presence of small motion which is typical of most MR studies, particularly brain studies.

### BRIEF DESCRIPTION

In accordance with one aspect of the invention, a method is provided. the method comprises acquiring an initial volumetric localizer to establish an initial object position and initial object orientation at an initial state, acquiring a fast localizer of the object at a present state, aligning the fast localizer to the initial volumetric localizers, determining object motion between the initial state and the present state, and modifying an imaging protocol based on the object motion.

In accordance with another aspect of the invention, a machine readable medium is provided. The machine readable medium comprises instructions configured to perform the previously described method.

In accordance with yet another aspect of the invention, an imaging system comprising the previously described machine readable medium is provided.

### DRAWINGS

These and other features, aspects, and advantages of the present invention will become better understood when the following detailed description is read with reference to the accompanying drawings in which like characters represent like parts throughout the drawings, wherein:

FIG. 1 illustrates an imaging system for generating digital images of an internal anatomy of a person, in accordance with an exemplary aspect of the present technique;

FIG. 2 is a flow diagram illustrating an operational flow in accordance with one embodiment of the invention;

FIG. 3 is a block diagram illustrating one embodiment of an image registration process for that aligning images;

FIG. 4 is a schematic diagram illustrating use of a transform T_{D} to generate a new scan plane in accordance with one embodiment;

FIG. 5 illustrates slices of an initial volumetric localizer image of a human brain;

FIG. 6 illustrates three fast 2D localizer images; and

FIG. 7 illustrates imaging results from a subject imaged by an MR scanner incorporating embodiments of the methods described herein.

### DETAILED DESCRIPTION

Embodiments of the present invention are generally directed to a system and method for patient motion compensation during magnetic resonance scans. Embodiments of the invention provide a method to quickly compute and refine a known patient position and orientation using a fast acquisition to collect necessary localizer data, and a fast registration method to update knowledge of anatomical scan planes.

In the following detailed description, numerous specific details are set forth in order to provide a thorough understanding of various embodiments of the present invention. However, those skilled in the art will understand that embodiments of the present invention may be practiced without these specific details, that the present invention is not limited to the depicted embodiments, and that the present invention may be practiced in a variety of alternative embodiments. In other instances, well known methods, procedures, and components have not been described in detail.

Furthermore, various operations may be described as multiple discrete steps performed in a manner that is helpful for understanding embodiments of the present invention. However, the order of description should not be construed as to imply that these operations need be performed in the order they are presented, nor that they are even order dependent. Moreover, repeated usage of the phrase “in one embodiment” does not necessarily refer to the same embodiment, although it may. Lastly, the terms “comprising”, “including”, “having”, and the like, as well as their inflected forms as used in the present application, are intended to be synonymous unless otherwise indicated.

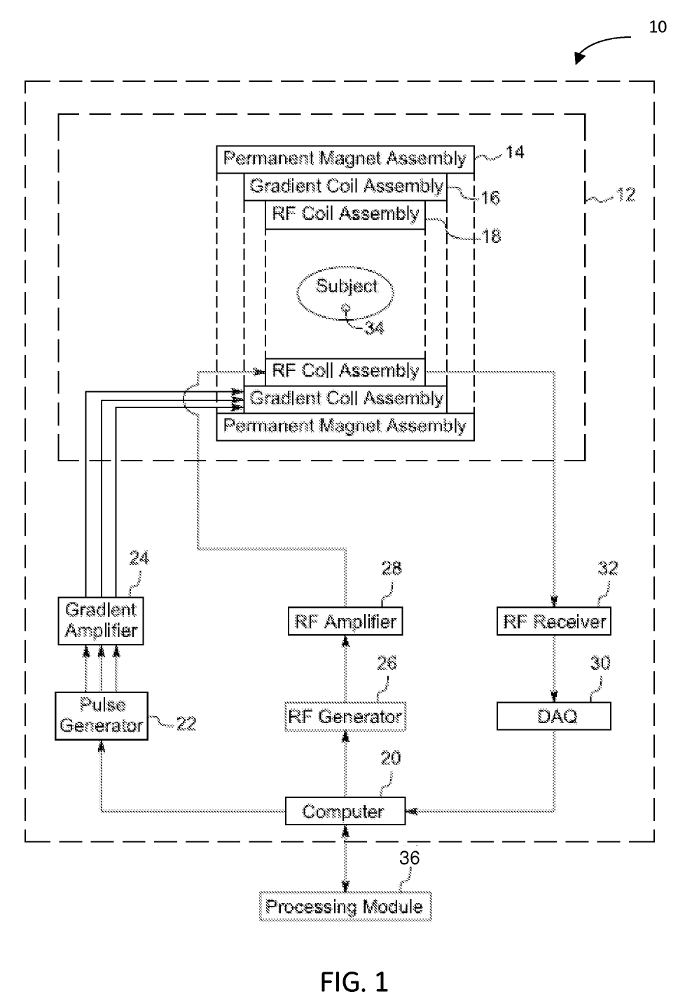

Referring now to FIG. 1, an imaging system 10 for generating digital images of an internal anatomy of a person, in accordance with an exemplary aspect of the present technique is illustrated. In the illustrated embodiment, the imaging system 10 is an MR imaging system is shown as including a scan unit 12, which is capable of scanning an object 34 and generating an image of an object 34 based on the magnetic resonance signals generated in the object 34 by emitting radio frequency (RF) pulses to the object 34 in a static magnetic field space. It may be noted that in one embodiment, the object 34 may include a patient. Although the present technique is described in terms of the object 34 including a patient, it may be noted that the present technique may also be applied to imaging other objects.

Moreover, although the exemplary embodiments illustrated hereinafter are described in the context of a MR imaging system, other imaging systems and applications such as industrial imaging systems and non-destructive evaluation and inspection systems, such as pipeline inspection systems, liquid reactor inspection systems, are also contemplated. Additionally, the exemplary embodiments illustrated and described hereinafter may find application in multi-modality imaging systems that employ an imaging system in conjunction with other imaging modalities, position-tracking systems or other sensor systems. Furthermore, it should be noted that the imaging system 10 may include imaging systems, such as, but not limited to, an X-ray imaging system, an ultrasound imaging system, a positron emission tomography (PET) imaging system, a computed tomography (CT) imaging system, or the like.

In the embodiment illustrated in FIG. 1, the imaging system 10 is shown as including a permanent magnet assembly 14, a gradient coil assembly 16, an RF coil assembly 18, a computer 20, a pulse generator 22, a gradient amplifier 24, an RF generator 26, an RF amplifier 28, a data acquisition unit 30, and an RF receiver 32. The permanent magnetic assembly 14 may include a pair of permanent magnets, for example. The pair of permanent magnets may form a static magnetic field in the imaging area in which the object 34 is carried. While imaging system 10 may include any suitable MRI scanner or detector, in the illustrated embodiment the system includes a full body scanner comprising a bore (not shown in Fig) into which a table (not shown) may be positioned to place an object 34 in a desired position for scanning. The static field may be formed such that the direction of the static field extends along a direction perpendicular to a direction of the bore axis. Scan unit 12 may be of any suitable type of rating, and may include scanners varying from 0.5 Tesla ratings to 1.5 Tesla ratings and beyond.

Scan unit 12 includes a series of associated coils for producing controlled magnetic fields, for generating radiofrequency excitation pulses, and for detecting emissions from gyromagnetic material within the object 34 in response to such pulses. A gradient coil assembly 16 is used for generating controlled magnetic gradient fields during examination sequences. An RF coil assembly 18 is provided for generating radiofrequency pulses for exciting the gyromagnetic material. In one embodiment, the permanent magnetic assembly 14 may be made of superconducting magnets.

Moreover, the pulse generator 22 may be configured to generate gradient signals. These gradient signals may be amplified by the gradient amplifier 24 and transmitted to the gradient coil assembly 16, in response to a control signal received from the computer 20. Additionally, in response, the gradient coil assembly 16 may be configured to produce magnetic field gradients in the scanning region, where the magnetic field gradients may be employed to aid in spatially encoding acquired signals.

In addition, the RF generator 26 may be configured to generate signals that are amplified by the RF amplifier 28 and transmitted to the RF coil assembly 18, in response to a control signal received from the computer 20. In response, the RF coil assembly 18 may be configured to generate RF signals that propagate through the object 34 in the scanning region. These RF signals propagating through the object 34 may in turn be configured to induce nuclei in predetermined regions of the object 34 to emit RF signals that may be received by the RF receiver 32. The received RF signals may then be digitized by the data acquisition unit 30. In one embodiment, the data acquisition unit 30 may employ a phase detector device to detect a phase of the magnetic resonance signals received by the RF coil assembly 18. Additionally, the data acquisition unit 30 may use an analog-to-digital converter (ADC) to convert analog magnetic resonance signals, into digital magnetic resonance signals.

The digitized signals may then be communicated to the computer 20. Computer 20 may be configured to direct the various components in the imaging system 10 to perform operations in correspondence with the scanning procedure. More particularly, the computer 20 may be configured to reconstruct an image slice corresponding to a slice of the object 34 from the acquired image data. The image so generated may then be displayed on a display device (not shown in FIG. 1) based on control signals received from the computer 20.

In accordance with further aspects of the present invention, the system 10 may include a processing module 36. The processing module 36 may be configured to perform automated scan planning using symmetry detection and image registration. More specifically, in one embodiment, processing module 36 may be configured to acquire an initial volumetric localizer to establish an initial object position and initial object orientation at an initial state, acquire a fast localizer of the object at a present state, align the fast localizer to the initial volumetric localizers to determine object motion between the initial state and the present state, and modify an imaging protocol using the object position and orientation at the present state. The processing module 36 may be implemented in hardware or as software and may be integrated as part of computer 20. In another embodiment, the processing module 36 may be located remotely from the imaging system 10 and may be communicatively coupled to the system 10 through a communications network.

Furthermore, the imaging system 10 may also include a storage unit (not shown in FIG. 1) that may be used to store data. In one embodiment, the storage unit may include memory configured to store the image data. It should be understood that any type of computer accessible memory device capable of storing the desired amount of data and/or code may be utilized by such an exemplary imaging system 10. Moreover, the storage unit may include one or more memory devices, such as magnetic, solid state, or optical devices, of similar or different types, which may be local and/or remote to the system 10. The storage unit may store data, processing parameters, and/or computer programs including one or more routines for performing the processes described herein.

With continuing reference to FIG. 1, in one exemplary embodiment, the computer 20 may be configured to generate two-dimensional (2D) digital images, three-dimensional (3D) digital images, or both 2D digital images and 3D digital images of the object to be imaged, such as of the internal anatomy of a patient, using the data received from the data acquisition unit 30.

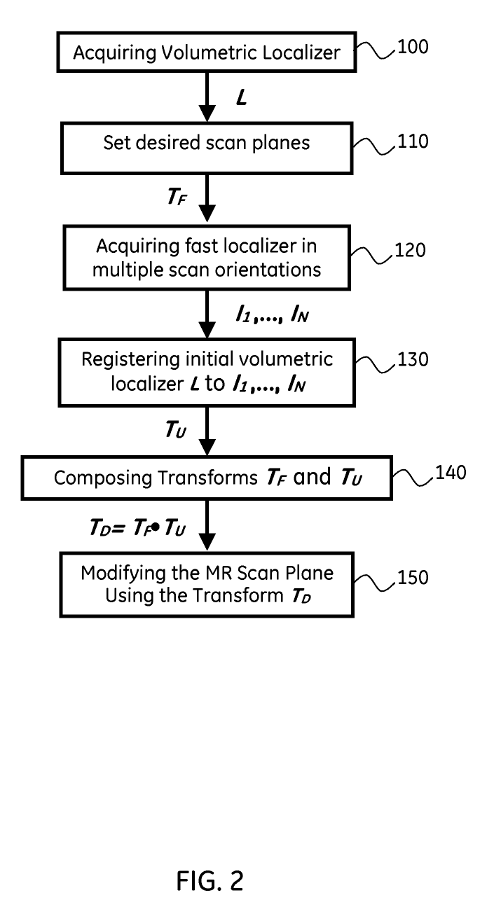

FIG. 2 is a flow diagram illustrating an operational flow in accordance with one embodiment of the invention. In block 100, a volumetric localizer is acquired to cover the entire object of interest. The localizer image is referred to as L, which is a function defined in three-dimensional space, L(x,y,z). In one embodiment, the desired scan planes are prescribed with respect to the object position and orientation as indicated by the localizer image L(x,y,z), (block 110). The scan plane prescription can be done automatically using a computer program, or manually by an MR scanner operator. Further, the scan planes are represented by a three-dimensional (3D) rigid transform, T_{F}. In accordance with one embodiment, before each series of diagnostic images is acquired, a set of fast two-dimensional localizer images or low resolution three-dimensional images are acquired, (block 120). The spatial locations and orientations of these (2D) or (3d) localizer images with respect to the scanner coordinate frame are known and the set of images are referred to as I₁, . . . , I_{N}.

Next, a registration algorithm is employed to align the initial volumetric localizer L(x,y,z) to images I₁, . . . , I_{N} with a 3D rigid transform, resulting in T_{U}, (block 130). The transforms T_{F} and T_{U} are then composed forming a transform T_{D} to provide information of the object position and orientation at present time (block 140), which can then be used by a computer, such as computer 20 and/or processing module 36 to prescribe the MR scan plane, (block 150).

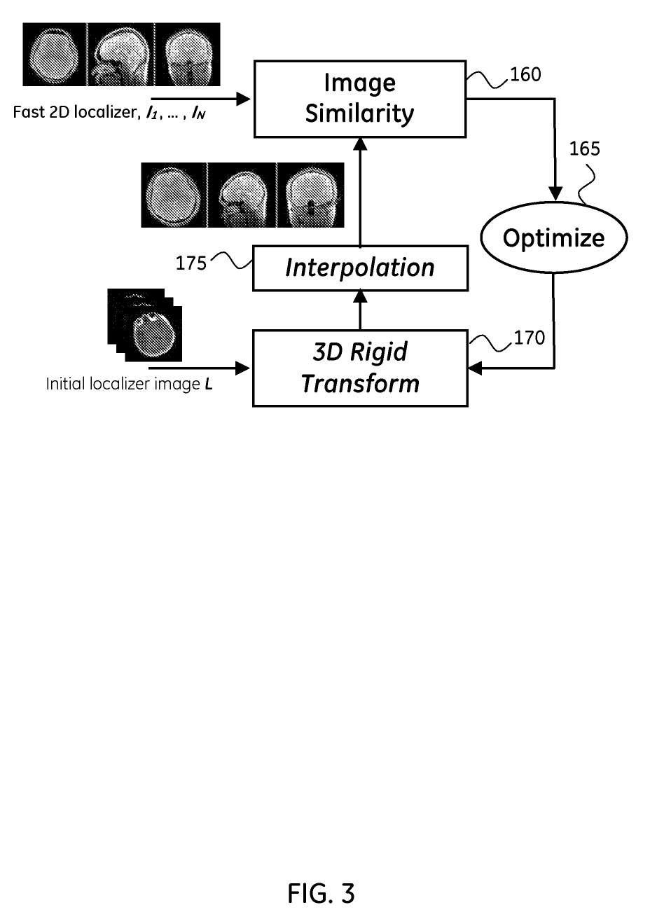

FIG. 3 is a block diagram illustrating one embodiment of an image registration process that aligns image L(x,y,z) with images I₁, . . . , I_{N}. In the illustrated embodiment, the image registration process is an iterative process through which an image similarity measure 160 is used to quantify the similarity between the image L(x,y,z) and the images I₁, . . . , I_{N}. The similarity measure may represent any of a number of known or yet to be developed similarity metrics including mutual information, cross-correlation, or a least-squares error metric. In one embodiment, an optimization process 165 is used to update the transform between the images, using the three-dimensional rigid transform 170 so as to maximize the similarity measure between image L(x,y,z) and images I₁, . . . , I_{N}. In one embodiment, in order to compute the image similarity measure 160, an interpolation step 175 is applied on the initial localizer image L under 3D rigid transform 170 to generate 2D images in the same spatial locations as the fast 2D localizers.

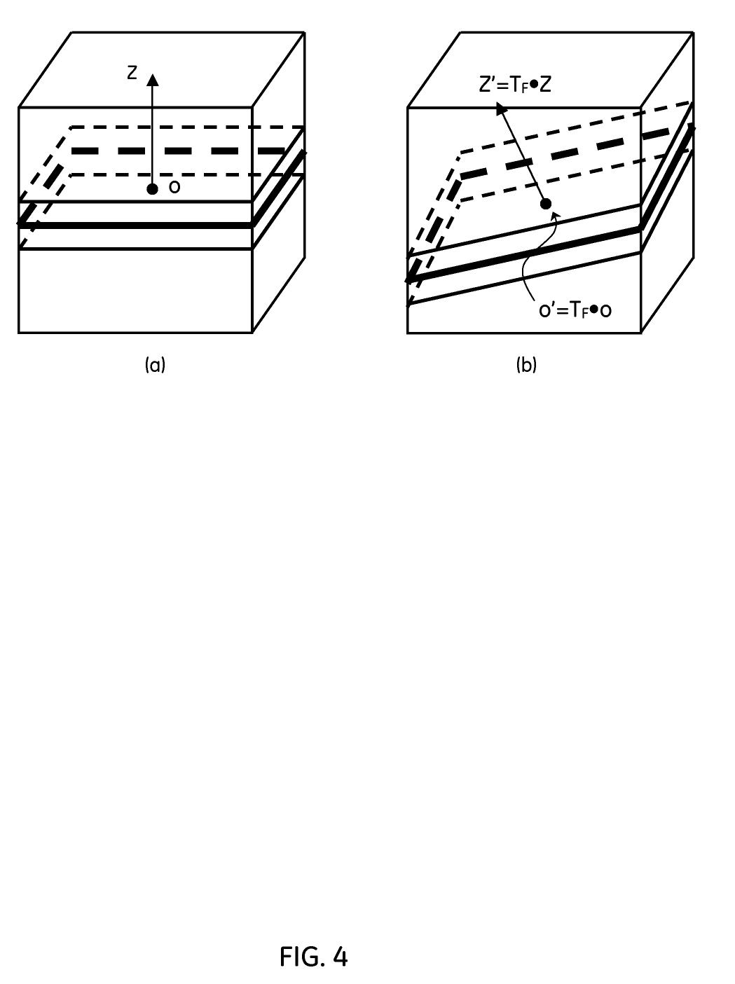

FIG. 4 illustrates one embodiment of how the composed transform T_{D} may be used to generate a new scan plane. For example, suppose a plane defined by a point o and a vector z is given in the anatomy space as shown by FIG. 4(a). The point corresponding to point o in the object space is o′=T_{D}·o; and the vector corresponds to z is z′=T_{D}·z. The point o′ and vector z′ define the scan plane in the object space.

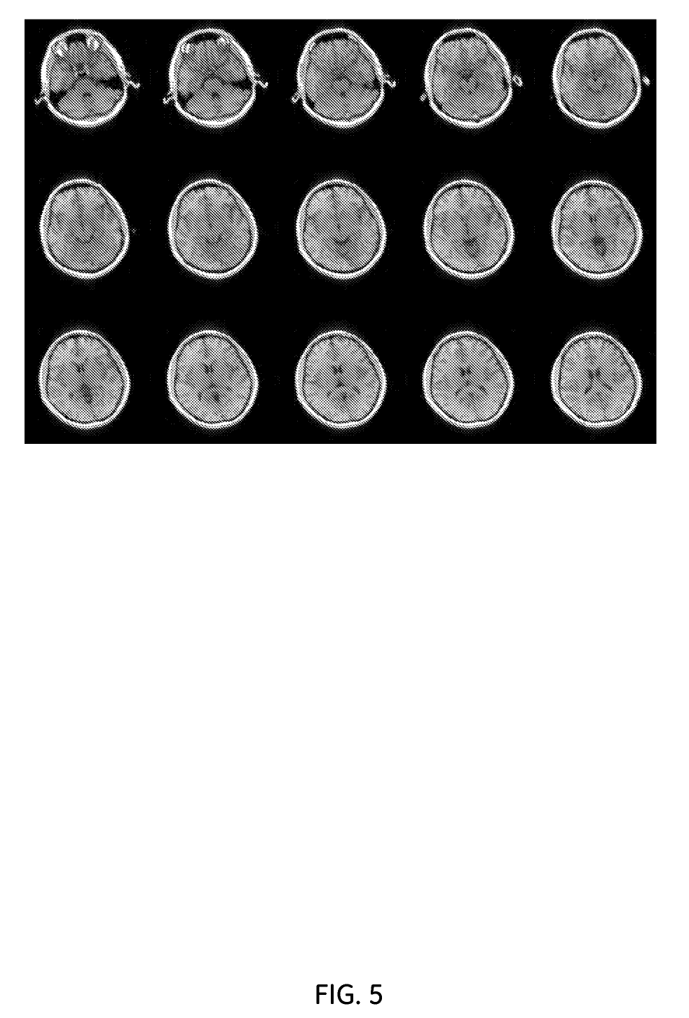

FIG. 5 illustrates 15 consecutive slices of an initial volumetric localizer image of a human brain.

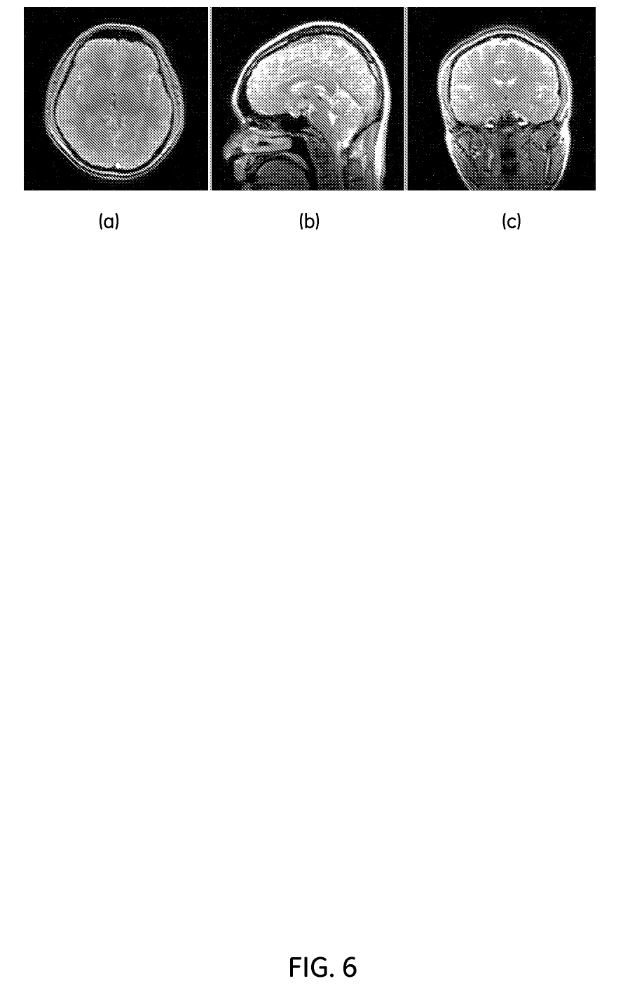

FIG. 6 illustrates three fast 2D localizer images in (a) axial, (b) sagittal, and (c) coronal orientations.

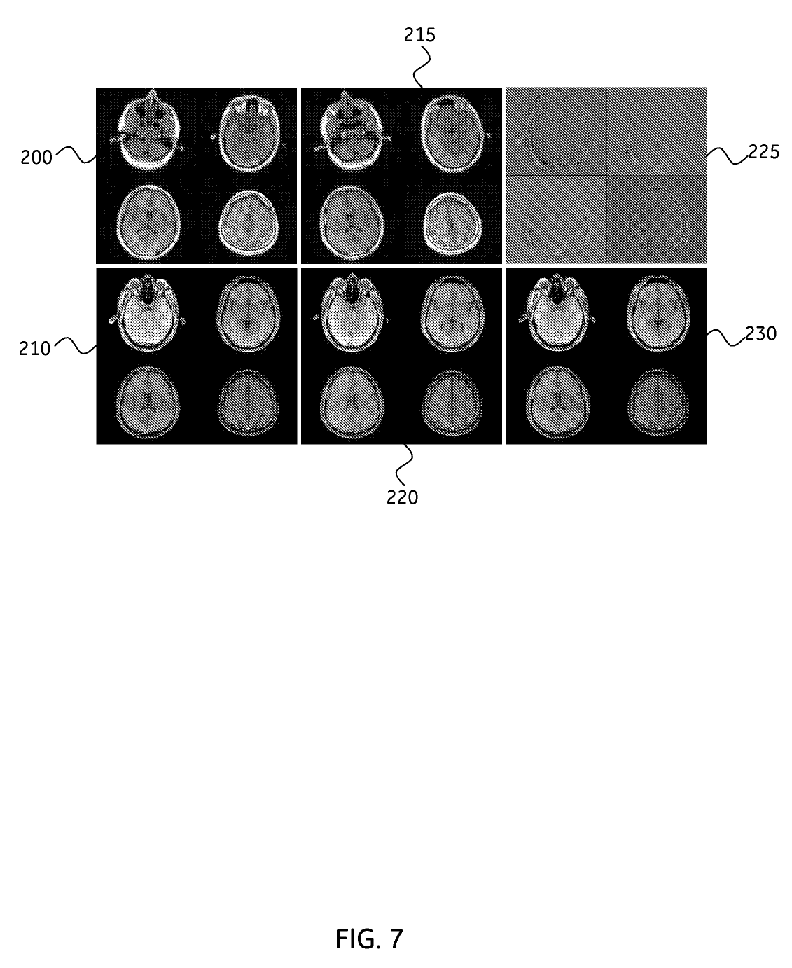

FIG. 7 illustrates imaging results from a subject imaged by an MR scanner incorporating embodiments of the methods described herein. The image block in the top-left (200) is a representative initial image of an original whole-head localizer. The image block on the bottom-left (210) illustrates standard axial images in initial head position. The top-middle image block (215) illustrates axial scout images in a new head position. The bottom-middle image block (220) illustrates standard axial images using fast registration from 2D localizer images. The top-right image block (225) illustrates the difference images between bottom-left image block 210 and the bottom-middle image block 220. Lastly, the bottom-right image block (230) illustrates standard axial images using full registration between the top-left image block 200 and the top-middle image block 215.

The above-description of the embodiments of the method for reconstructing an image and the system for reconstructing an image have the technical effect of improving workflow by enhancing image quality and reducing image artifacts, thereby allowing acceleration of image processing applications.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A method comprising:
acquiring an initial volumetric localizer to establish an initial object position and initial object orientation at an initial state;
acquiring a fast localizer of the object at a present state;
aligning the fast localizer to the initial volumetric localizers;
determining object motion between the initial state and the present state; and
modifying an imaging protocol based on the object motion.

2. The method of claim 1, further comprising acquiring an image using the modified protocol.

3. The method of claim 1, wherein the initial object position and the initial object orientation are known

4. The method of clam 1, wherein the volumetric localizer is aligned with the fast localizer using a rigid transform.

5. The method of claim 1, wherein acquiring a fast localizer comprises acquiring a two-dimensional localizer or a three-dimensional localizer.

6. The method of claim 5, wherein acquiring a fast localizer comprises acquiring a plurality of image slices in a plurality of orientations.

7. The method of claim 1, further comprising applying a known transformation between the initial object position and the present object position to a location and orientation of scan planes to produce new scan planes corresponding to the current object position and current object orientation.

8. The method of claim 1, further comprising acquiring a fast localizer prior to each image acquisition subsequent to the initial volumetric localizer to eliminate positional shifts due to object motion.

9. A machine readable medium comprising instructions stored therein, which when executed cause a processor to
acquire an initial volumetric localizer to establish an initial object position and initial object orientation at an initial state;
acquire a fast localizer of the object at a present state;
align the fast localizer to the initial volumetric localizers to determine object motion between the initial state and the present state; and
modify an imaging protocol using the object position and orientation at the present state.

10. The machine readable medium of claim 9, wherein the instructions are further configures to acquire a two-dimensional localizer.

11. The machine readable medium of claim 10, wherein the instructions are configured to acquire a plurality of image slices in a plurality of orientations.

12. The machine readable medium of claim 9, wherein the instructions are further configured to apply a known transformation between the initial object position and the present object position to a location and orientation of scan planes to produce new scan planes corresponding to the current object position and current object orientation.

13. The machine readable medium of claim 9, wherein the instructions are further configured to acquire an image using the modified protocol.

14. The machine readable medium of claim 9, wherein the instructions are further configured to acquire a fast localizer prior to each image acquisition subsequent to the initial volumetric localizer to eliminate positional shifts due to object motion.

15. An imaging system comprising the machine readable medium of claim 9.

16. The imaging system of claim 15, wherein the imaging system comprises a magnetic resonance imaging system.
